(19) United States
(12) Patent Application Publication
(10) Pub. No.: US 2013/0266614 A1
(43) Pub. Date: Oct. 10, 2013

(54) LIPID CARRIER FOR DELIVERY OF BIOACTIVE SUBSTANCE AND PHARMACEUTICAL COMPOSITION

(71) Applicants: Kuang-Wen Liao, Hsinchu City (TW); Yen-Ku Liu, Taipei City (TW); Chia-Hung Chen, Taichung City (TW)

(72) Inventors: Kuang-Wen Liao, Hsinchu City (TW); Yen-Ku Liu, Taipei City (TW); Chia-Hung Chen, Taichung City (TW)

(21) Appl. No.: 13/443,455

(22) Filed: Apr. 10, 2012

(51) Int. Cl. A61K 9/00; A61K 38/43; A61K 39/395; A61K 38/16

(52) U.S. Cl. 424/400; 514/1.1; 424/94.1; 424/130.1

(57) ABSTRACT

The present invention provides a lipid carrier for delivering of a bioactive substance. The lipid carrier includes a lipid layer, a positive charged polymer and a surface active polymer. The positive charged polymer and the surface active polymer are respectively distributed on the lipid layer by non-covalent bonds. The present invention also provides a pharmaceutical composition. The present invention is advantageous for delivering bioactive substance efficiently.

### BACKGROUND OF THE INVENTION

1. Field of Invention

The present invention relates to a lipid carrier and a pharmaceutical composition, and in particular, to a lipid carrier and a pharmaceutical composition capable of absorbing a bioactive substance.

2. Related Art

Liposome is a micro hollow sphere with lipid bilayer and has a structure similar to that of a cell membrane. Particularly, the inner layer and the outer layer of the liposome are formed by the hydrophilic end of the lipid as hydrophilic aqueous phase systems, while the hydrophobic end of the lipid is aggregated into a lipophilic group, forming a surrounded structure between the inner layer and the outer layer. This structure allows the self-assembling to form a vesicle without adding any other surface active agent. A great space in the core of the liposome can be used to carry the hydrophilic substance, while the internal of the lipid bilayer can be used to encapsulate the hydrophobic substance. Therefore, it is possible to encapsulate hydrophilic and hydrophobic medicines at the same time, and this feature makes the applications of the liposome wider.

The structure of liposome is similar to a cell membrane. The liposome has the properties of excellent biocompatibility and biodegradability, and has no bio-toxicity, so that it is widely used in biological technology, such as transfection, drug delivery, vaccine, and gene therapy. In other words, the materials, such as nucleic acid, protein, and drugs are encapsulated in the liposome, such that the materials enter an organism together with the liposome, so as to achieve the purpose of transfection and drug delivery. If the liposome is used to delivery toxic drug, it further reduces the toxicity and the undesired side effects of the drug used. Therefore, for using the liposome to encapsulate the substances, there is a significantly effect to prolong the circulation time of substances in body.

Otherwise, it is well known that there are many proteins involving in the normal physiological function in cells of organisms and closely related to many diseases. Hence, the protein engineering for therapy is developed and improved to transport a normal protein into cells and replace the defective protein in organism, so as to achieve the directly and safely therapeutic effect. However, the therapeutic efficacy and the application scope are restricted by the delivery efficiency and the retaining activity of the protein after transported. In the liposome transporting techniques as mentioned above, when encapsulating macromolecules such as proteins, especially active proteins, it still exists a bottleneck in rapidly forming a complex compound including the proteins and carriers. In general, this process should be performed by chemical modification or with a long reaction time (more than one hour). Thus, it is a burden for the production cost of the liposome transporting techniques.

In addition, the conventional liposome transporting technique has a problem of poor retained activity percentage of the transported substance, and it is a restriction of clinical application and hard to be a stable and spread carrier.

Hence, it is an important issue to provide a lipid carrier that can be easily and rapidly manufactured, has a great delivery efficacy, and retains high activity after the protein is transported to the target site, so as to improve the therapeutic efficacy in the clinical therapy and broaden the application scope by utilizing the protein drugs.

### SUMMARY OF THE INVENTION

In view of the foregoing, the purpose of the present invention is to provide a lipid carrier that can be easily and rapidly manufactured, has a great delivery efficacy, and retains high activity after the protein is transported to the target site, so as to improve the therapeutic efficacy in the clinical therapy and broaden the application scope for utilizing the bioactive substance as drugs.

To achieve the purpose as described above, the present invention discloses a lipid carrier for delivering a bioactive substance, which is absorbed to the lipid carrier. The lipid carrier includes a lipid layer, a positive charged polymer and a surface active polymer. The positive charged polymer and the surface active polymer are respectively distributed on the lipid layer by non-covalent bonds.

In one embodiment of the present invention, the ratio of the lipid layer, the positive charged polymer and the surface active polymer ranges between 3:1:1 and 60:1:1.

In one embodiment of the present invention, the composition of the lipid layer includes DLPC, DOPC, DMPC, DPPC, DSPC, DMPE, DPPE, DOPE, DMPA, DPPA, DOPA, DMPG, DPPG, DOPG, DMPS, DPPS, or DOPS.

In one embodiment of the present invention, the positive charged polymer includes polyamine, polyethyleneimine, polyvinylpyrrolidone or polyacetic acid.

In one embodiment of the present invention, the surface active polymer includes crosslinked polyacrylate, saponin or polyethylene glycerol.

In one embodiment of the present invention, the bioactive substance is a pharmaceutically activated protein molecule.

In one embodiment of the present invention, the bioactive substance is an enzyme, an antibody, a hormone, a transcription factor or a translation factor.

In one embodiment of the present invention, the positive charged polymer is distributed on an outer surface of the lipid layer, and the bioactive substance is absorbed on the outer surface of the lipid layer by non-covalent bonds between the positive charged polymer and the bioactive substance.

In one embodiment of the present invention, the non-covalent bond is an electrostatic force or a hydrogen bond.

In one embodiment of the present invention, the positive charged polymer forms a hook structure on an outer surface of the lipid layer, and the bioactive substance is fixed to the lipid layer by engaging with the hook structure.

In one embodiment of the present invention, the hook structure is originated from a branch of the positive charged polymer.

In one embodiment of the present invention, the weight percentage of the bioactive substance relative to the lipid layer ranges from 20% to 50%.

In addition, the present invention also discloses a pharmaceutical composition including a lipid layer, a positive charged polymer, a surface active polymer and a bioactive substance. The positive charged polymer and the surface active polymer are respectively distributed on the lipid layer by non-covalent bonds. The bioactive substance is absorbed to the lipid layer.

In one embodiment of the present invention, the ratio of the lipid layer, the positive charged polymer, and the surface active polymer ranges between 3:1:1 and 60:1:1.

In one embodiment of the present invention, the composition of the lipid layer includes DLPC, DOPC, DMPC, DPPC, DSPC, DMPE, DPPE, DOPE, DMPA, DPPA, DOPA, DMPG, DPPG, DOPG, DMPS, DPPS, or DOPS.

In one embodiment of the present invention, the positive charged polymer includes polyamine, polyethyleneimine, polyvinylpyrrolidone, or polyacetic acid.

In one embodiment of the present invention, the surface active polymer includes crosslinked polyacrylate, saponin or polyethylene glycerol.

In one embodiment of the present invention, the bioactive substance is a pharmaceutically activated protein molecule.

In one embodiment of the present invention, the bioactive substance is an enzyme, an antibody, a hormone, a transcription factor or a translation factor.

In one embodiment of the present invention, the positive charged polymer is distributed on an outer surface of the lipid layer, and the bioactive substance is distributed on the outer surface of the lipid layer by non-covalent bonds forming with the positive charged polymer.

In one embodiment of the present invention, the non-covalent bond is an electrostatic force or a hydrogen bond.

In one embodiment of the present invention, the positive charged polymer forms a hook structure on an outer surface of the lipid layer, and the bioactive substance is fixed to the lipid layer by engaging with the hook structure.

In one embodiment of the present invention, the hook structure is originated from a branch of the positive charged polymer.

In one embodiment of the present invention, the weight percentage of the bioactive substance relative to the lipid layer ranges from 20% to 50%.

In summary, a lipid carrier and a pharmaceutical composition in accordance with the present invention for delivering a bioactive substance have a distinctive structure, that is substantially the combination of a lipid layer, a positive charged polymer and a surface active polymer binding by non-covalent bonds, and is able to efficiently absorb the bioactivity substance to the surface of the carrier through the manufacturing process of the lipid carrier. Moreover, the lipid carrier and pharmaceutical composition in accordance with the present invention is appropriate to enhance the fixation of the bioactivity substance on the lipid carrier and provide a certain protection by the distinctive structure configured by the three compositions as mentioned above. Therefore, it has the advantages of high biocompatibility of the conventional liposome and targeting the bioactive substance to a specifically therapeutic site (e.g. the specific portion to be therapeutically treated). Besides, because of the protection provided by the structure of the lipid carrier, it has the advantages of reducing the harmful influences on the bioactive substance during the circulation or transporting process and increasing the retained percentage of the bioactive substance and prolonging the period of its activity.

Concretely speaking, comparing to the conventional art, the lipid carrier of the present invention can carry the bioactive substance to be delivered just by absorbing means, instead of the complicated encapsulating means. Therefore, it is unnecessary to modify the lipid carrier with chemical functional groups, such that it not only saves the process time but improves the clinical application, thereby providing excellent cost-effectiveness. Certainly, the pharmaceutical composition composed of the lipid carrier of the present invention can avoid the activity of the bioactive substance from diminishing, so it has better efficacy and broader scope of application.

### BRIEF DESCRIPTION OF THE DRAWINGS

The invention will become more fully understood from the detailed description and accompanying drawings, which are given for illustration only, and thus are not limitative of the present invention, and wherein:

FIG. 1 shows the structure of the lipid carrier for delivering the bioactive substance according to an embodiment of the present invention;

FIG. 2A to FIG. 2C show experimental results of the time, amount and the energy change of the lipid carrier incorporated BSA, respectively;

FIG. 3A to FIG. 3D show experimental results of the surface of lipid carrier with and without the incorporation of BSA using AFM analysis; and

FIG. 4A to FIG. 4D show experimental results of the enzymatic activity of beta-glucuronidase delivered by lipid carrier to the HepG2 cells.

### DETAILED DESCRIPTION OF THE INVENTION

The present invention will be apparent from the following detailed description, which proceeds with reference to the accompanying drawings, wherein the same references relate to the same elements.

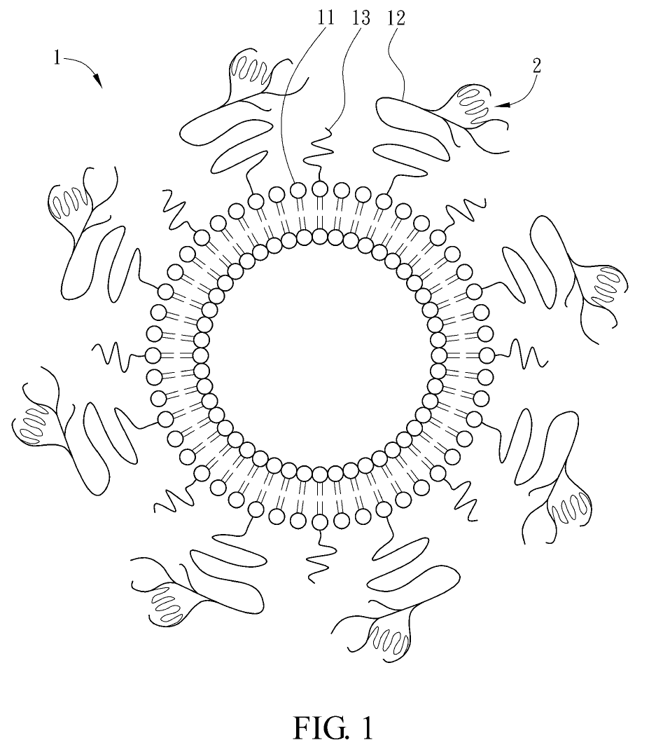

FIG. 1 shows the structure of a lipid carrier 1 for delivering the bioactive substance according to an embodiment of the present invention. As shown in FIG. 1, the lipid carrier 1 includes a lipid layer 11, a positive charged polymer 12 and a surface active polymer 13. In this case, the positive charged polymer 12 and the surface active polymer 13 are respectively distributed on the lipid layer 11 by non-covalent bonds. Following, the lipid carrier 1 will be firstly described in the specification.

In one embodiment of the present invention, the lipid layer 11 has a lipid bilayer structure and is mainly composed of neutral lipid. The composition of the neutral lipid includes, for example but not limited to, DLPC (dilinoleoylphosphatidylcholine), DOPC (dioleoylphosphatidylcholine), DMPC (dimyristoylphosphatidylcholine), DPPC (dipalmitoylphosphatidylcholine), DSPC (disaturatedphosphatidylcholine), DMPE (dimyristoylphosphatidylethanolamine), DPPE (1,2-Bis-(diphenylphosphino)ethane), DOPE (dioleoylphosphatidyl ethanolamine), DMPA (dimethylolpropionic acid), DPPA (diphenylphosphoryl azide), DOPA (dioleoylphosphatidic acid), DMPG (dimyristoylphosphatidylglycerol), DPPG (dipalmitoylphosphatidylglycerol), DOPG (dioleoyl phosphatidylglycerol), DMPS (dimyristoylphosphatidylserine), DPPS (dipalmitoylphosphatidylserine), or DOPS (dioleoylphosphatidylserine). In a preferred aspect, the lipid layer 11 is composed substantially by DLPC and DOPC. In another embodiment of the present invention, a fluorescent dye can be added during the manufacturing process of the lipid carrier, such that the formed lipid layer 11 is equipped with the fluorescent property and can be used to easily track the composition.

When the lipid carrier 1 is provided close to the cell in organism, the lipid layer 11 will fuse with the cell membrane of the cell and enter the cell by the opposite properties of the hydrophilicity and the hydrophobicity. The term “cell in organism” herein preferably refers to any cell in an organism, or a cell line cultured in vitro. The organism as mentioned above mainly includes mammals, such as mouse, human, bovine, goat, swine, monkey, canine, or feline, and preferably is human. The cell preferably is the therapeutic mammal cell such as the human breast cancer cell.

Because the lipid layer 11 has the structure including an inner layer and an outer layer, the positive charged polymer 12 and the surface active polymer 13 can be distributed on the inner or outer layer by non-covalent binds in the process of forming the lipid layer 11. Since the formation rate of non-covalent bonds is faster than that of the covalent bonds and has lower energy threshold, it is beneficial to improve the manufacturing efficiency. The term “non-covalent bond” used herein includes the hydrophilic interaction, hydrophobic interaction, electrostatic interaction, hydrogen bond, van der waals force, or the combinations thereof.

The positive charged polymer 12 refers to the positively charged long-chain polymers, such as polyamine, PEI (polyethylenimine), polyvinylpyrrolidone, or polyacetic acid. The surface active polymer 13 can be for example but not limited to crosslinked polyacrylate, saponin or polyethylene glycerol (PEG). In this embodiment, the positive charged polymer 12 is PEI, and the surface active polymer 13 is PEG.

It should be noted that the ratio of the lipid layer 11, the positive charged polymer 12 and the surface active polymer 13 ranges between 3:1:1 and 60:1:1. In this embodiment, the ratio of the lipid layer 11, the positive charged polymer 12 and the surface active polymer 13 ranges between 10:3:3 and 60:1:1, preferably, between 10:3:3 and 30:1:1, and more preferably, is 3.3:1:1.

The lipid layer 11, the positive charged polymer 12 and the surface active polymer 13 can construct a closed form lipid carrier 1 in a special ratio, and the lipid carrier 1 can have for example but not limited to a sphere shape, football shape or other three-dimensional irregular shape. In this embodiment, the lipid carrier 1 can be referred to a liposome.

The above-mentioned materials are mixed with the special ratio in a vessel, and then treated with vortex or added by other materials so as to form the lipid carrier 1 of the present invention. In detail, a lipid solution containing the neutral lipids aforementioned is added to a pear-shaped flask. Next, the solvent in the lipid solution is removed by vacuum concentration to remain a multi-layer, film-like neutral lipid layer 11 in the bottom of the vessel. Then, a solution containing the positive charged polymer 12 and a solution containing the surface active polymer 13 are added to the same vessel to contact with the lipid layer 11. Similarly, the positive charged polymer 12 and the surface active polymer 13 can be any one as described above.

To distribute evenly the positive charged polymer 12 and the surface active polymer 13 on the lipid layer 11 to form the lipid carrier 1, the solution containing the lipid layer 11, the positive charged polymer 12 and the surface active polymer 13 can be shaken by manual or mechanical means, so as to form a closed form, sphere shaped carrier structure with a space in the center. The positive charged polymer 12 and the surface active polymer 13 has the hydrophobic and hydrophilic properties, and thus, they can bind to the lipid layer 11 by non-covalent bonds. Furthermore, in another embodiment, the formed lipid layer 1 can be passed through a pore membrane to obtain a plurality of uniform-sized lipid carriers 1. This feature can keep high consistency in subsequent applications. In this embodiment, the size of the lipid carrier 1 is between 2 to 400 nm, and preferably, smaller than 100 nm. It needs to be noted that the non-covalent bond is formed by the hydrophilic interaction, hydrophobic interaction, electrostatic interaction, hydrogen bond, van der waals interaction, or the combinations thereof. It also needs to be noted that, except for the pore membrane, the lipid carrier 1 can be also purified by for example but not limited to centrifugation.

As mentioned above, the positive charged polymer 12 and the surface active polymer 13 can be embedded in the lipid layer 1 due to their physical properties instead of formation of any chemical bonds. Thus the lipid carrier 1 is manufactured by simply mixing and shaking, so that it is possible to simplify the process steps and shorten the process time thereof.

The suitable bioactive substance 2 absorbed to the lipid carrier 1 of the present invention is universal, but preferably, includes for example but not limited to an enzyme, an antibody, a hormone, a transcription factor, a translation factor, or other substances that perform physiological or biochemical functions in organism to induce variant biological reaction.

In detail, as shown in FIG. 1, the absorbed bioactive substance 2 can be, for example, a targeting substance such as antibody, cytokine, peptide with specific sequence or nucleic acid with specific sequence. Thus, the lipid carrier 1 has cells specificity, tissue specificity, or tumor specificity. In other words, the lipid carrier 1 can target or be limited in a specific tissue or surrounding the cells to be treated by the specific recognition between the substances as mentioned above and their binding partners to improve the transporting efficiency or the therapy effect of the lipid carrier 1. Moreover, the specificity of the lipid carrier 1 can reduce harmful effect on the normal tissues or cells, so the lipid carrier 1 of the present invention can be an excellent drugs carrier and gene carrier.

Certainly, in more preferable embodiment, the targeting substance can be linked to the positive charged polymer 12 by chemical modification, and the lipid carrier 1 absorbs, for example, a therapeutic substance, such as enzyme, antibody, hormone, transcription factor, or translation factor. With this structure, the lipid carrier 1 has not only transport specificity, but also therapeutic effect.

Since the means of linking the targeting substance are apparent to persons skilled in the art, the detailed description is omitted herein. However, the solution containing the targeting substance can be added, for example but not limited to, when the lipid layer 11 is mixed with the positive charged polymer 12 and the surface active polymer 13, or after the lipid carrier 1 is formed.

For the process of absorbing the bioactive substance 2, the lipid layer 11, the positive charged polymer 12 and the surface active polymer 13 can be simultaneously mixed with the bioactive substance 2, so that the bioactive substance 2 is directly absorbed to the surface of the lipid carrier 1.

Owing to the special composition of the lipid carrier 1 of the present invention, the bioactive substance 2 can be absorbed to the positive charged polymer 12 by non-covalent bonds. In addition, a force is applied to stabilize the binding thereof. First of all, the non-covalent bond is formed by electrostatic interaction and hydrogen bond, which is caused by the spacial distribution of the electricity on the surface of the lipid carrier 1, so that the bioactive substance 2 with negative charge tends to be restricted here. Another force can be caused by a plurality of tiny branches formed from the positive charged polymer 12 on the lipid layer 11 for constituting a stable velcro-like coupling structure with the bioactive substance 2. The tiny branches of the positive charged polymer 12 can be a hook-shaped, a button-shaped structure, or the likes. With the different types of the bioactive substance 2, the micro structure of the bioactive substance 2 can be coupled with the tiny branches of the positive charged polymer 12, in substance, by snapping, embedding, or hooking under the principle of forming a stable structure, such that the bioactive substance 2 is fixed on the surface of the lipid carrier 1. Accurately speaking, the bioactive substance 2 stably absorbed on the lipid carrier 1 is by the interaction of the non-covalent bonds and the three-dimensional structure formed with the positive charged polymer 12.

Because the lipid layer 11, the positive charged polymer 12 and the surface active polymer 13 tend to form a larger lipid carrier 1, and the bioactive substance 2 is stably absorbed to the lipid carrier 1, it is beneficial to absorb larger size of the bioactive substance 2, increasing the binding capacity of the lipid carrier 1 per unit, meanwhile, expanding the application scope. In addition, the lipid carrier 1 of the present invention can be directly mixed with the bioactive substance 2 to form a pharmaceutical composition, and therefore, it has the features of simplifying the process step and shortening the process time.

Therefore, in a preferable embodiment in accordance with the present invention, after the bioactive substance 2 is absorbed to the lipid carrier 1 by the means of mixing as mentioned above, the weight percentage of the bioactive substance 2 relative to the lipid carrier 1 ranges from 20% to 50%. As to the high binding capacity, the lipid carrier 1 of the present invention absorbs the bioactive substance 2 by non-covalent bonds, so as to form an overlapping multi-layer of the bioactive substance 2. On the other hand, because of the non-covalent bonds, the activity or the conformation of the absorbed bioactive substance 2 can be avoided from being influenced or destroyed. Additionally, it has a characteristic that the bioactive substance 2 can be rapidly and stably absorbed on the lipid carrier 1. When the bioactive substance 2 is continuously absorbed on the lipid carrier 1 until the absorption saturation is reached, the absorbed bioactive substance 2 can form a three-dimensional structure as a barrier and then prevent other substances from being absorbed in sequential process. Thus, the possibility of replacement of the absorbed bioactive substance 2 is relative low. Base on this property, the lipid carrier 1 of the present invention has high purity and high efficiency of absorbing the bioactive substance 2.

Of course, in order to label or track the site of the composition of the present invention, the lipid carrier 1 can further be linked with conjugates. The conjugates can be for example but not limited to a chromogenic substance or a radioactive substance.

The present invention also provides a pharmaceutical composition constituted by the lipid carrier 1 as mentioned above absorbing the bioactive substance 2. Since the absorbed bioactive substance 2 on the lipid carrier 1 can fulfill, provide, or inhibit a physiological pathway, the composition of the present invention has a pharmaceutical value. The constitution and the features of the pharmaceutical composition of the present invention are illustrated in the embodiments as described above.

Hereafter, The following experimental examples demonstrates the function of the lipid carrier of the present invention to absorb the bioactive substances, and the operative process and achievement of the lipid carrier to deliver the bioactive substance to the living cells. It should be noted that, the following description is to illustrate the present invention in detail, so that those skilled in the art can implement the present invention, but is not intended to limit the scope of the present invention.

### Experimental Example 1

### Protein Binding Ability of LIPID CARRIER Devoid of PEI or PEG

Forty micrograms of each lipid carrier is incubated with excess BSA (over 100 μg) to occupy the unreacted sites of the lipid carrier for 20 min at 37° C. After removal of any unbound BSA by centrifugation, the pellet is washed with 1 ml deionized water.

Lipid carrier is incubated at 4° C., 25° C. and 37° C. for up to 5 days. At each time point, the protein binding capacity of lipid carrier (40 μg) is measured by incubating the lipid carrier with 200 μg of BSA for 20-30 min at 37° C. Following washes and resuspension, the amount of bound BSA is measured as described above.

Binding of BSA to Lipid Carrier

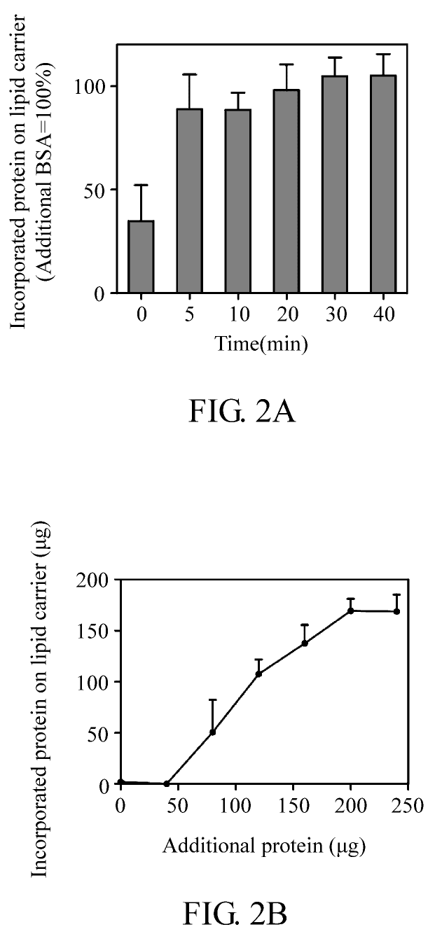

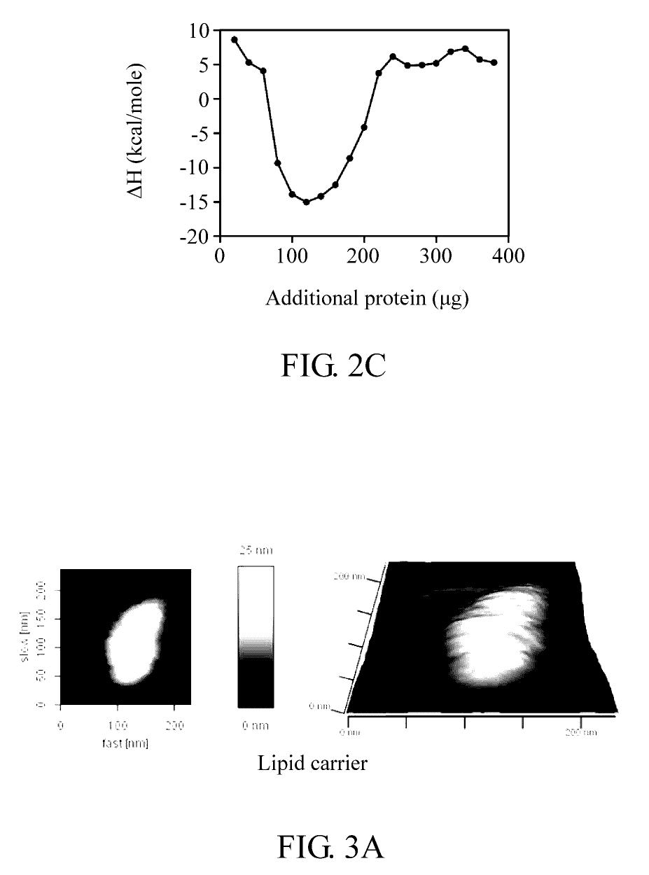

Sequentially, this experimental example examines whether lipid carrier is able to capture proteins using BSA as a reference. First, the experimental example demonstrates that after adding BSA to lipid carrier and incubating over time at 37° C., the maximal binding is achieved within 30 min, as shown in FIG. 2A.

The binding capacity of lipid carrier (40 μg in 1 ml) for BSA incorporation following a 20 min incubation is then determined. The result as shown in FIG. 2B shows that the amount of BSA incorporation can be saturated in a concentration-dependent manner. The maximal binding capacity is 168.6±16.4 μg of BSA to 40 μg of lipid carrier or a binding ratio about 4:1 (w/w).

Second, the experimental example investigates the energy released using an ITC measurement while mixing lipid carrier with BSA. The result as shown in FIG. 2C reveals that heat is released starting at a critical concentration of BSA (40 μg), indicating that BSA binding to lipid carrier began at this point and terminated after 200 μg BSA is added. Interestingly, this coincides with the results from FIG. 2B.

According to the results of the present experimental example, it obviously indicates that the lipid carrier of the present invention absorbing proteins is faster than for encapsulating, that is, the efficiency of the lipid carrier bind the proteins by absorbing can be significantly improved.

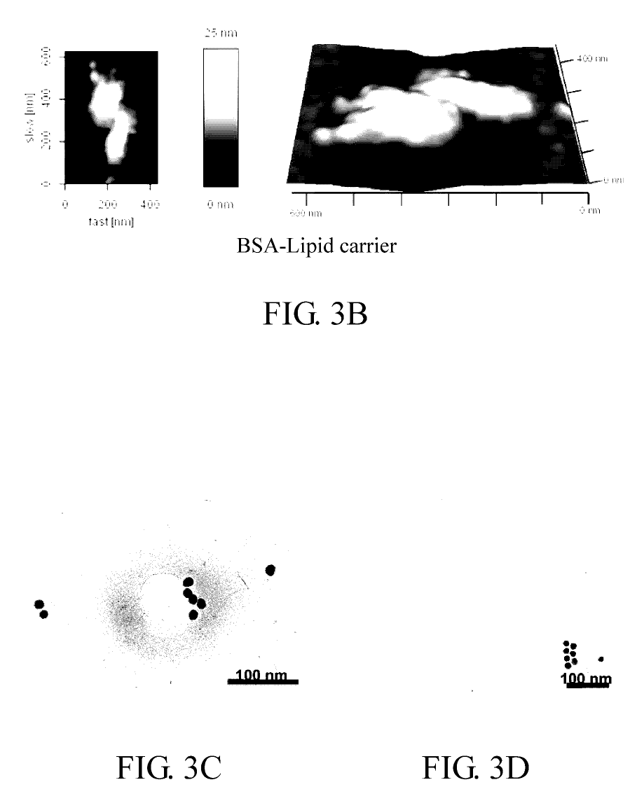

Finally, the present experimental example, investigates the surface of lipid carrier with and without the incorporation of BSA using AFM analysis. As shown in FIGS. 3A and 3B, the BSA-lipid carrier complex possesses a rough and raised surface relative to the smooth surface of lipid carrier alone. The present experimental example proves that the captured BSA is located at or near the surface of experimental example. To further visualize the incorporated BSA, BSA-conjugated gold particles are utilized as markers. As shown in FIGS. 3C and 3D, it shows that BSA-gold particles are located on surface of lipid carrier using TEM without negative staining but not on lipid carriers without polymers. This indicates that the proteins might be bound through polymers and that the binding occurs on the surface of the lipid carrier.

### Experimental Example 2

The present experimental example demonstrates the retained activity percentage of the bioactive substance after the lipid carrier absorbs the bioactive substance and is transported into cells.

Enzymatic Activity of Transported Protein

HepG2 is seeded in a 24-well plate at 1×105 per well in 1 ml DMEM medium containing 10% FBS and 1% PSA overnight. Before the treatment, medium will be removed, washed, and then well is added with 1 ml DMEM medium without serum. One microliters of LPPC (10 mg/ml) is mixed with 37.5 μg of beta-glucuronidase (PG; kindly provided by Dr. T L Cheng of Kaoshung Medical School, Taiwan) in 10 μl of final volume at 25° C. for 30 min. Following, 10 μl of complex is added into each well at 37° C. for 4 hour, and the cell is then washed with PBS for three times. The cell is fixed with 4% paraformaldehyde (v/v) in PBS for 20 min and washed three times by PBS, treat with 0.25% triton X-100 (v/v) in PBS and washed three times again. The 20 μl of X-gluc (5 mg/ml) is added into the each well which containing 1 ml PBS at 37° C. for 12 hour, and the well is observed and photographed in a visible light field by microscopy (IX70-S1F2; Olympus).

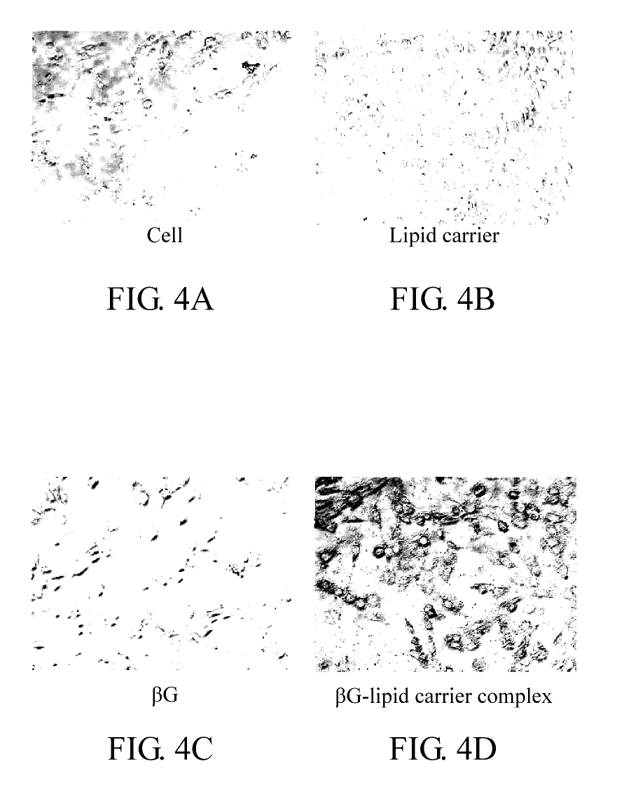

In the present experimental example, βG (beta-glucuronidase) is utilized as the bioactive substance. After the bioactive substance forms a complex with lipid carrier of the present invention, the complex is incubated with HepG2 for a period of time, then, the βG substrate, X-glucose, is added. As shown in FIG. 4, the group labeled βG-lipid carrier is treated with the composition as described above, and the cells are dyed green (shown as dark areas in FIG. 4) in the group, it indicates that the bioactive substance exactly can be transported to the cells by being absorbed to the lipid carrier, and still active in cells as well.

Certainly, the bioactive substance aforementioned can be also replaced with other pharmaceutical or immunogenic substances, and the experimental examples will be still demonstrated the same results.

In summary, a lipid carrier and a pharmaceutical composition in accordance with the present invention for delivering a bioactive substance have a distinctive structure, that is substantially the combination of a lipid layer, a positive charged polymer and a surface active polymer binding by non-covalent bonds, and is able to efficiently absorb the bioactivity substance to the surface of the carrier through the manufacturing process of the lipid carrier. Moreover, the lipid carrier and pharmaceutical composition in accordance with the present invention is appropriate to enhance the fixation of the bioactivity substance on the lipid carrier and provide a certain protection by the distinctive structure configured by the three compositions as mentioned above. Therefore, it has the advantages of high biocompatibility of the conventional liposome and targeting the bioactive substance to a specifically therapeutic site (e.g. the specific portion to be therapeutically treated). Besides, because of the protection provided by the structure of the lipid carrier, it has the advantages of reducing the harmful influences on the bioactive substance during the circulation or transporting process and increasing the retained percentage of the bioactive substance and prolonging the period of its activity.

Concretely speaking, comparing to the conventional art, the lipid carrier of the present invention can carry the bioactive substance to be delivered just by absorbing means, instead of the complicated encapsulating means. Therefore, it is unnecessary to modify the lipid carrier with chemical functional groups, such that it not only saves the process time but improves the clinical application, thereby providing excellent cost-effectiveness. Certainly, the pharmaceutical composition composed of the lipid carrier of the present invention can avoid the activity of the bioactive substance from diminishing, so it has better efficacy and broader scope of application.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A lipid carrier for delivering a bioactive substance, wherein the bioactive substance is absorbed to the lipid carrier, the lipid carrier comprising:
a lipid layer;
a positive charged polymer distributed on the lipid layer by non-covalent bonds; and
a surface active polymer distributed on the lipid layer by non-covalent bonds.

2. The lipid carrier of claim 1, wherein the ratio of the lipid layer, the positive charged polymer and the surface active polymer ranges between 3:1:1 and 60:1:1.

3. The lipid carrier of the claim 1, wherein the composition of the lipid layer comprises DLPC, DOPC, DMPC, DPPC, DSPC, DMPE, DPPE, DOPE, DMPA, DPPA, DOPA, DMPG, DPPG, DOPG, DMPS, DPPS, or DOPS.

4. The lipid carrier of the claim 1, wherein the positive charged polymer comprises polyamine, polyethyleneimine, polyvinylpyrrolidone or polyacetic acid.

5. The lipid carrier of the claim 1, wherein the surface active polymer comprises crosslinked polyacrylate, saponin or polyethylene glycerol.

6. The lipid carrier of the claim 1, wherein the bioactive substance is a pharmaceutically activated protein molecule.

7. The lipid carrier of the claim 1, wherein the bioactive substance is an enzyme, an antibody, a hormone, a transcription factor or a translation factor.

8. The lipid carrier of the claim 1, wherein the positive charged polymer is distributed on an outer surface of the lipid layer, and the bioactive substance is absorbed on the outer surface of the lipid layer by the non-covalent bonds between the positive charged polymer and the bioactive substance.

9. The lipid carrier of the claim 8, wherein the non-covalent bond is an electrostatic force or a hydrogen bond.

10. The lipid carrier of the claim 1, wherein the positive charged polymer forms a hook structure on an outer surface of the lipid layer, and the bioactive substance is fixed to the lipid layer by engaging with the hook structure.

11. The lipid carrier of the claim 10, wherein the hook structure is originated from a branch of the positive charged polymer.

12. The lipid carrier of the claim 1, wherein the weight percentage of the bioactive substance relative to the lipid layer ranges from 20% to 50%.

13. A pharmaceutical composition, comprising:
a lipid layer;
a positive charged polymer distributed on the lipid layer by non-covalent bonds;
a surface active polymer distributed on the lipid layer by non-covalent bonds; and
a bioactive substance.

14. The pharmaceutical composition of claim 13, wherein the ratio of the lipid layer, the positive charged polymer, and the surface active polymer ranges between 3:1:1 and 60:1:1.

15. The pharmaceutical composition of claim 13, wherein the composition of the lipid layer comprises DLPC, DOPC, DMPC, DPPC, DSPC, DMPE, DPPE, DOPE, DMPA, DPPA, DOPA, DMPG, DPPG, DOPG, DMPS, DPPS, or DOPS.

16. The pharmaceutical composition of claim 13, wherein the positive charged polymer comprises polyamine, polyethyleneimine, polyvinylpyrrolidone, or polyacetic acid.

17. The pharmaceutical composition of claim 13, wherein the surface active polymer comprises crosslinked polyacrylate, saponin or polyethylene glycerol.

18. The pharmaceutical composition of claim 13, wherein the bioactive substance is a pharmaceutically activated protein molecule.

19. The pharmaceutical composition of claim 13, wherein the bioactive substance is an enzyme, an antibody, a hormone, a transcription factor or a translation factor.

20. The pharmaceutical composition of claim 13, wherein the positive charged polymer is distributed on an outer surface of the lipid layer, and the bioactive substance is absorbed on the outer surface of the lipid layer by the non-covalent bonds forming with the positive charged polymer.

21. The pharmaceutical composition of claim 20, wherein the non-covalent bond is an electrostatic force or a hydrogen bond.

22. The pharmaceutical composition of the claim 13, wherein the positive charged polymer forms a hook structure on an outer surface of the lipid layer, and the bioactive substance is fixed to the lipid layer by engaging with the hook structure.

23. The pharmaceutical composition of the claim 22, wherein the hook structure is originated from a branch of the positive charged polymer.

24. The pharmaceutical composition of the claim 13, wherein the weight percentage of the bioactive substance relative to the lipid layer ranges from 20% to 50%.
